(19) United States
(12) Patent Application Publication
(10) Pub. No.: US 2009/0318983 A1
(43) Pub. Date: Dec. 24, 2009

(54) METHOD AND APPARATUS FOR DETECTION AND TREATMENT OF AUTONOMIC SYSTEM IMBALANCE

(76) Inventors: Antonis A. Armoundas, Lincoln, MA (US); Ki H. Chon, Mount Sinai, NY (US)

Correspondence Address: CHOATE, HALL & STEWART LLP, TWO INTERNATIONAL PLACE, BOSTON, MA, 02110, US

(21) Appl. No.: 12/373,227

(22) PCT Filed: Jul. 6, 2007
(86) PCT No.: PCT/US07/72889

§ 371 Date: Jan. 9, 2009

Related U.S. Application Data
(60) Provisional application No. 60/819,716, filed on Jul. 10, 2006.

(51) Int. Cl. A61N 1/362; A61M 37/00

(52) U.S. Cl. 607/3; 607/17

(57) ABSTRACT

Method and apparatus for preventing autonomic system disturbances by recording physiological parameters, measuring beat-to-beat variability of these parameters, and using the measured beat-to-beat variability to control the delivery of drug therapy and electrical impulses to the heart.

This application claims priority to U.S. provisional application Ser. No. 60/819,716 filed Jul. 10, 2006, the contents of which are incorporated herein by reference.

### BACKGROUND OF THE INVENTION

Cardiovascular disease is the greatest cause of morbidity and mortality in the industrialized world. It not only strikes down a significant fraction of the population without warning but also causes prolonged suffering and disability in an even larger number. Sudden cardiac death (SCD) is prevalent in the population, but it is difficult to treat because it is difficult to predict in which individuals it will occur, and it often occurs without warning, in an out of hospital setting. It has been widely agreed that the implantable cardioverter defibrillator (ICD) reduces the incidence of SCD in high risk patients.

### Autonomic Nervous System and Susceptibility to Arrhythmias

The major purpose of the sympathetic nervous system is to maintain cardiac function on a short-term basis. Although it has been reported that physiological levels of cathecholamines induce after depolarizations in normal ventricular myocytes¹, it becomes evident that conditions and activities associated with intense sympathetic activity such as, for example, trauma and sports are rarely associated with ventricular tachycardia/fibrillation (VT/VF) in the normal heart, or alternatively, the survival advantage conferred by the powerful compensatory properties of the sympathetic nervous system would not be harnessed. Superscript numbers refer to the references appended hereto. The contents of all of these references are incorporated herein by reference. On the other hand, when the long-term sympathetic nervous system is needed to meet the demands of organ perfusion due to, for example, reduced myocardial contractility, then the same system becomes maladaptive increasing the susceptibility of the heart to life threatening VT/VF. Thus, any process that inhibits or limits intense sympathetic activity duration without altering the mechanical (contractile) capacity of the heart could provide critical protection against such pro-arrhythmic effects.

The effects of the sympathetic nervous system are complicated by the interactions with other neuroendocrine systems that affect electrophysiological properties, such as the rennin angiotensin system and the parasympathetic system². The parasympathetic nervous system activity exerts an anti-sympathetic effect that is likely to significantly inhibit the proarrhythmic effects of the sympathetic nervous system. Evidence for this assertion comes from animal experiments in which analysis of heart rate and blood pressure signals under control conditions indicates that the parasympathetic dominates at rest³, and inhibits the action of the sympathetic nervous system. However, the inhibitory effects of the parasysmpathetic activity are transient and vanish several minutes after parasympathetic activity diminishes. When parasympathetic activity is absent the sympathetic nervous system effects are not only exacerbated due to loss of parasympathetic activity, but sympathetic activity is self-promoting its activity that inhibits parasympathetic activity. This sympathetic nervous system inhibition of the parasympathetic activity depends on the duration and intensity of the prior sympathetic nervous system activity⁴⁻⁶. This may be at least one of the mechanisms by which parasympathetic activity is chronically reduced in patients with chronic left ventricular dysfunction⁷ and one of the mechanisms that promotes elevated sympathetic nervous system activity.

The association among chronic elevation in sympathetic nervous system activity, myocardial dysfunction, susceptibility to VT/VF and sudden cardiac death has been recognized for several years^{8, 9}. There are many mechanisms by which the sympathetic nervous system can alter cardiac electrophysiological properties that could precipitate susceptibility to VT/VF. These mechanisms are exacerbated, for example, by myocardial ischemia resulting from increased oxygen demand due to increased heart rate, contractility and other factors, or by myocardial stretch as a consequence of increased blood pressure. As a result, long-term processes reduce sympathetic nervous activity such as, for example, in chronic heart failure in which β-adrenergic receptor down-regulation has been observed¹⁰. These long-term processes probably impart important protection at the cellular level but they do not reduce sympathetic nervous system activity to normal levels, nor do they eliminate susceptibility to VT/VF and sudden cardiac death.

Overall, β-blockers have provided unsatisfactory protection against sudden cardiac death^{11, 12}, and there are several possible explanations for the incomplete protection of β-blocker therapy: (i) the sympathetic nervous system does not participate in all ventricular tachyarrhythmic events, (ii) β-blocker concentration at the effector sites is inadequate due to pharmacokinetic interference, inadequate dose or lack of specificity, and (iii) there is inappropriate blocking of the sympathetic activity. Furthermore, there is often underprescription or underdosing¹³ because of concern for adverse effects including cardiac decompensation, reduced exercise tolerance, brady-arrhythmias, and reduced libido. Such underprescription or underdosing may also affect the effectiveness of β-blockers that makes necessary the investigation of alternative strategies.

### Autonomic Imbalance Assessment

An effective approach in tracking the time-varying variation in sympathetic nervous system activity is based on the application of power spectral techniques on the RR intervals, blood pressure, instantaneous-lung-volume (respiration), muscle sympathetic nerve activity and other parameters^{14, 15}.

From the time that variability in the RR intervals was first appreciated as a harbinger of sudden cardiac death in post myocardial infarction patients by Wolf et al.¹⁶, numerous studies¹⁷⁻²⁰ have established a significant relationship between the variability in the RR intervals and susceptibility to lethal ventricular arrhythmias. A major challenge has been to describe variability in the RR intervals mathematically. The phenomenon of fluctuations in the interval between consecutive heart beats has been the subject of investigations using a wide range of methodologies including time domain^{21, 22}, frequency domain²²⁻²⁴, geometric²⁵, and non-linear²⁶⁻²⁸, methods. With the general recognition of nonlinear dynamics theory in the mid 1980's, it was proposed that the variability in the RR intervals should be viewed as the result of nonlinear determinism in the regulatory systems governing the heart rate. Parameters indicative of possible low-dimensional nonlinear determinism include Lyapunov exponents, strange attractors and correlation dimensions.²⁷⁻²⁹

Thus, consistent with autonomic imbalance, patients who have suffered a myocardial infarction have a marked decrease in the variability in the RR intervals due to a decrease in vagal and an increase in sympathetic neural activities. Power spectral analysis of the variability in the RR intervals has been shown to be useful in risk stratification after myocardial infarction³⁰. Power spectra of the variability in the RR intervals can be divided into three main frequency zones: the power spectral density (PSD) below 0.04 Hz is considered to be very low frequency (VLF), between 0.04 Hz and 0.15 Hz is low frequency (LF), and between 0.15 HZ and 0.5 HZ is high frequency (HF). The LF is found to be mediated by both the sympathetic and parasympathetic nervous influences and the HF is unequivocally believed to be dominated solely by the parasympathetic nervous system³¹. The VLF has been proven to be related to factors other than the autonomic nervous system (ANS) (e.g. temperature, hormones etc.)³². The ratio of the LF to HF power obtained from spectral analyses has been shown to be a good marker of the sympathovagal balance in assessing the variability in the RR intervals³². For example, a large LF/HF ratio suggests predominantly sympathetic control, whereas a small LF/HF ratio indicates predominantly vagal control. However, the LF/HF ratio for clinical utility has not gained wide acceptance, mainly because it is an approximation of the autonomic balance and does not truly reflect the balance of the two nervous influences. This stems from the fact that the LF/HF ratio assumes the LF is mediated by the sympathetic nervous system, despite the prevailing understanding that the LF reflects both the sympathetic and parasympathetic nervous systems.

Another deficiency of the LF/HF ratio is that the method is linear and does not properly account for nonlinear characteristics of the ANS. A plethora of recent studies has shown that the physiological mechanisms responsible for heart-rate fluctuations have nonlinear components³³. While the LF/HF ratio obtained via the PSD is inaccurate, it shows great promise of being a useful non-invasive marker for determining the state of the autonomic nervous system, if a new method is developed that can separate the dynamics of the two nervous systems. This new method should also account for the nonlinear components of heart rate fluctuations, in order to more accurately represent the nonlinear properties of heart rate dynamics.

Recently we developed and validated a novel mathematical technique to analyze electrocardiographic RR signals to accurately isolate sympathetic and parasympathetic components of the autonomic nervous system activity, known as Principal Dynamic Modes (PDM). Our analyses reveal that the first two dominant PDMs obtained from the RR time series of healthy human subjects correspond to the two autonomic nervous activities. This has been demonstrated in both time and frequency domains. The application of the autonomic nervous blocking agents, propranolol and atropine, corroborated our finding that the magnitude of the waveforms corresponding to either sympathetic or parasympathetic nervous activities were significantly reduced. Furthermore, the method specifically accounts for the inherent nonlinear dynamics of heart rate control, which the PSD does not. Overall, the significance of this technique is based on the fact that no other method has been able to achieve such result to date. Thus, the ability to accurately separate dynamics of sympathetic and parasympathetic nervous activities has many clinical applications. For example, there are many disease states that are initiated, affected or exacerbated because of imbalances of the two components of the autonomic nervous system. The PDM algorithm is simple to implement and can be modified to provide on- and off-line analysis for many and different diagnostic and therapeutic clinical applications.

### The Implantable Cardioverter Defibrillator

The ICD is an implantable device (shown in FIGS. 1a and 1b) that detects the initiation of arrhythmias, such as ventricular tachycardia or fibrillation, and terminates them by delivery of one or more electrical impulses to the heart. Often the energy of these impulses is quite large compared to the energy of impulses delivered by an artificial pacemaker which is used to pace the heart but not to terminate arrhythmias. The increased ease of ICD implantation as well as advances in ICD technology has led to a rapid growth in the rate of ICD implantation. However, ICDs generally are used to terminate an arrhythmia, such as ventricular tachycardia or fibrillation, only after the arrhythmia has started. This feature of ICD function may lead to patients losing consciousness once the arrhythmia starts and also leads to patients experiencing what may be very uncomfortable electrical discharges of the ICD. Frequent ICD discharge can lead to extreme psychological stress in many patients. Some patients have an ICD placed, only to suffer recurrent shocks and finally to have the device deactivated³⁴. Recently, it was shown that a rapid and progressive electrophysiological deterioration occurs during ventricular fibrillation that may explain the decreased probability of successful resuscitation after prolonged fibrillation⁹. Also, the more often the ICD discharges, the shorter is the life of its battery. Frequent ICD discharge can also damage the heart tissue itself and as a result may make the heart more susceptible to future arrhythmias. Thus it would be highly desirable to be able to be able to prevent arrhythmias from starting rather than terminating them after their initiation by administration of an electrical shock.

While ICDs currently are an effective therapy for the termination of heart rhythm disturbances³⁵⁻³⁷, their role is to deliver electrical impulses to terminate the arrhythmia rather than to prevent its onset. Thus, patients are being subjected to a serious arrhythmia for a period of time until therapy is delivered. Also, delivery of electrical impulses from the ICD may be painful and may damage the heart. To date there has been no way to prevent arrhythmias from initiating rather than treating them with what may be much higher energy electrical pulses after the arrhythmias have been initiated.

### SUMMARY OF THE INVENTION

This invention involves method and apparatus for preventing heart rhythm disturbances comprising: recording cardiac electrical activity, measuring beat-to-beat variability in the cardiac electrical activity, and using the beat-to beat variability to control therapy and reduce the likelihood of occurrence of heart rhythm disturbances. In one preferred embodiment, these aspects can be implemented in an implantable device that detects changes in the autonomic tone (reflected by the individual contributions of the sympathetic and parasympathetic systems) and delivers therapy by means of electrical pulses or low-energy shocks to alter the abnormal autonomic tone.

In one preferred embodiment the RR interval variability of ECG signals is mathematically analyzed to accurately isolate sympathetic versus parasympathetic components of autonomic nervous system activity, known as Principal Dynamic Modes (PDM). This capability of the PDM algorithm is significant because to date, no other method has been able to achieve such a result. Specifically, the better accuracy obtained with the PDM approach compared to the spectral approach is because the PDM approach is able to separate out the dynamics of the two nervous activities, whereas the LF/HF ratio estimated using the spectral approach suffers from the assumption that the low frequency power is solely due to sympathetic activity. The ability to accurately separate dynamics of sympathetic and parasympathetic nervous activities has many clinical applications, such as providing appropriate pacing stimuli to restore the balance of sympathetic and parasympathetic systems prior to VT/VF, without hemodynamically compromising the heart. In another preferred embodiment the therapy is the delivery of a chemical substances. In another preferred embodiment the therapy is the delivery of electrical impulses to the heart. In another preferred embodiment the electrical impulses are controlled to alter the variability in the diastolic interval. In another preferred embodiment the heart rhythm disturbance is a tachyarrhythmia. In another preferred embodiment the heart rhythm disturbance is a bradyarrhythmia. In another preferred embodiment the beat-to-beat electrical activity of the heart is recorded from a passive electrode within the heart. In another preferred embodiment the beat-to-beat arterial blood pressure is recorded from a passive electrode at the arterial tree. In another preferred embodiment the instantaneous lung volume is recorded. In another preferred embodiment the physiological signal of instantaneous lung volume (respiration) is estimated by at least one other recorded physiological signal. In another preferred embodiment the measuring of the beat-to-beat variability is performed by an implanted device. In another preferred embodiment the therapy is delivered by an implanted device. In another preferred embodiment, the implanted device serves as a cardiac pacemaker or a cardiac cardioverter/defibrillator. In another preferred embodiment, the implantable device has means for generating electrical stimulating pulses of specified energies and applying the pulses to body tissue at specified times. In another preferred embodiment, the measuring of beat-to-beat variability further involves identifying periods when there is an increased probability that a heart rhythm disturbance may occur. In another preferred embodiment, therapy is delivered during the periods of increased probability that a heart rhythm disturbance may occur.

In another preferred embodiment the measuring of the beat-to-beat variability in electrocardiographic waveform, blood pressure and instantaneous lung volume is performed by means outside the body. In another preferred embodiment, therapy is delivered during the periods of increased disturbance of the sympathetic and parasympathetic physiological balance.

### BRIEF DESCRIPTION OF THE DRAWINGS

FIGS. 1a and b are schematic views of an implantable cardioverter defibrillator in the human torso.

FIG. 2 is a schematic view of an implanted device with drug ports.

FIGS. 3a and 3b are graphs of the dynamics of parasympathetic and sympathetic (lower right panel) during supine position.

FIG. 4 is a block diagram showing an algorithm for application of pharmaco-therapy and electrical-therapy.

FIG. 5(a) is a diagram showing electrocardiographic characteristics.

FIG. 5(b) is a graph showing mean (u) and standard deviation (u) of the rate of the pacing stimuli.

FIG. 5(c) is a graph showing the relationship between the standard-deviation of the distribution of the timings of the pacing stimuli with respect to the end of the T-wave of the N^{th} beat (T_{end}^{N}).

FIG. 5(d) is a diagram of an example of the sensing algorithm; following detection of the QRS complex a “post sense refractory period” will be employed. Then, the sensing algorithm will use an auto-gain algorithm and a different sensitivity to detect the T-wave.

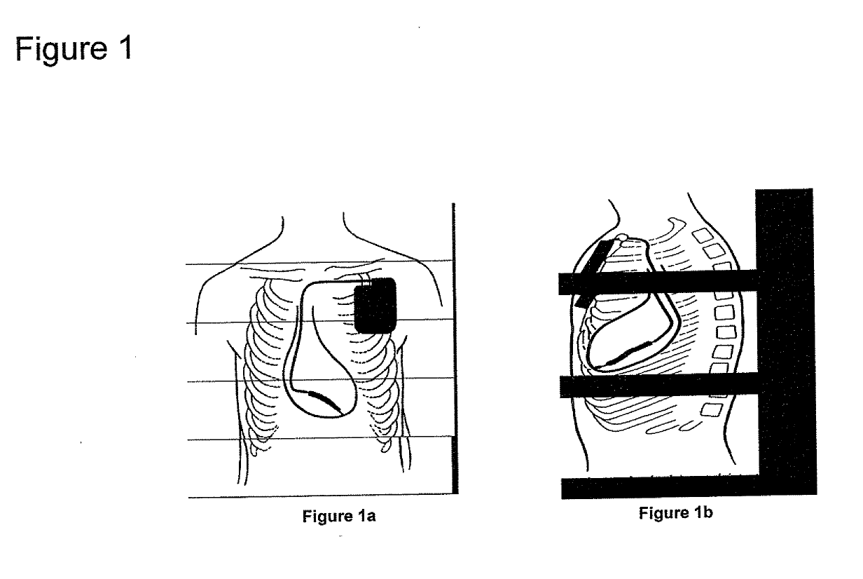

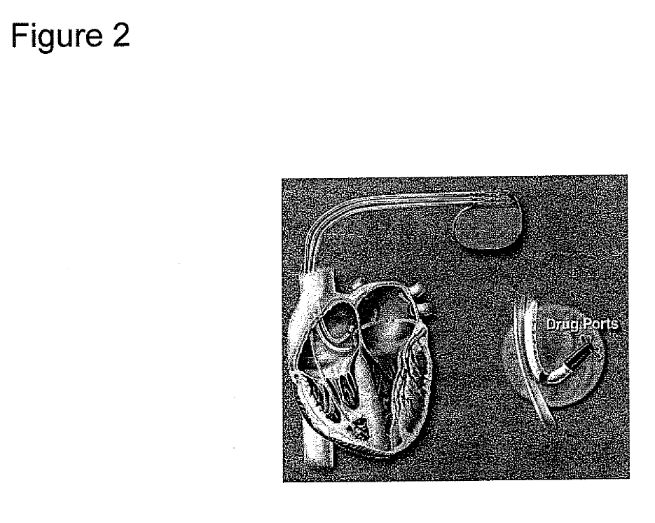

### DESCRIPTION OF THE PREFERRED EMBODIMENT

In a preferred embodiment the electrical activity of a subject's heart is sensed by electrodes (“sensing electrodes” or “passive electrodes”) in or on the patient's heart or electrodes located elsewhere in or on the subject's body. Measurements of beat-to-beat cardiac activity will be continuously monitored and compared with the patient's baseline measurement values. When the patient specific criteria are indicative of abnormal autonomic activity these measurements will trigger therapy.

In one preferred embodiment the device will measure beat-to-beat variability in the timing (RR interval) and/or morphology of the ventricular ECG waveforms, that we call autonomic index (AI)^{16, 19-29, 38-40}. To determine the AI, we will use the method known as the PDM. The PDM is a method based on extracting only the principal dynamic components of the signal via eigendecomposition. The PDMs are calculated using Volterra-Wiener kernels based on expansion of Lagurre polynomials. Among all possible choices of expansion bases, there are some that require the minimum number of basis functions to achieve a given mean-square approximation of the system output. This minimum set of basis functions are termed the PDM of the nonlinear system. This method specifically accounts for the inherent nonlinear dynamics of heart rate control, which the current method of power spectral density is unable to do. A minimum set of basis functions is determined using a method widely known as principal component analysis, in which the dominant eigenvector and eigenvalues are retained as they relate more closely to the true characteristics of the signal and non-dominant eigenvectors and eigenvalues represent noise or nonessential characteristics. Thus, principal component analysis is an approach to separate only the essential dynamic characteristics from a signal that is corrupted by noise. We have modified the PDM technique to be used with even a single output signal of HRV data, whereas the original PDM required both input and output data. A summary of the procedure is presented.

The accurate estimation of the Volterra-Wiener kernel requires a signal with broadband spectral characteristics. In many instances, the HR data do not exhibit broadband characteristics. Instead, significant power exists in the very low frequency (VLF) of the HR data compared to LF and HF. Consequently, the spectral power bands of interest, the LF and HF, are dwarfed by the significantly higher spectral power in the VLF band. An approach we took to reduce high spectral power in the VLF band is the method introduced by Tarvainen⁴¹, with the aim of reducing VLF power to the level of the LF and HF bands so that overall spectral characteristics are broadband. The result of this broadening process is labeled as HRc. The PDM method requires both the input and output data, but since we have only the output signal of HR recordings, we need to create an input signal with broadband spectral characteristics. We use normalized HRc, delayed by one unit, as the input, and undelayed HRc as the output signal to estimate the Volterra-Wiener kernel. Eigendecomposition of the kernel gives eigenvalues and eigenvectors, and then a set of eigenvectors is selected according to the absolute value of respective eigenvalues to reconstruct the output signal, which is then subtracted from the HRc signal to obtain estimation error, labeled HRe. In a preferred embodiment, the criterion for selecting the set of eigenvectors is that they account for our set threshold value of 90% of the HR dynamics. The created signal, HRe, is considered to be the input signal, which has the broadband characteristics needed for accurate estimation of PDMs. The obtained input data, HRe, is then normalized to a unit variance (HRn). HRn is used as an input signal and HRc is used as an output signal to estimate the Volterra-Wiener kernel. The eigendecomposition of this kernel gives eigenvectors, which are the final values of the PDMs, and eigenvectors then represent the relative importance of each PDM. The two most dominant PDMs are selected to represent the dynamics corresponding to the sympathetic and parasympathetic nervous activities. The technical details of estimating the Volterra-Wiener kernel and extracting the PDMs from the kernel are described in subsequent paragraphs.

While the obtained PDMs are in time-domain representation, we convert the PDMs to the frequency domain via the Fast Fourier transform (FFT) to facilitate validation of the two ANS activities, as they are usually illustrated in the frequency domain. Therefore, hereupon, we will describe the PDMs' dynamic characteristics in the frequency domain.

The technical specification involving the methodology of the PDM is as follows. In discrete time, the general input-output relation of a stable (finite-memory) nonlinear time-invariant dynamic system is given by the discrete-time Volterra series:

$$y ( n ) = {k}_{0} + {\sum }_{m = 1}^{M} {k}_{1} ( m ) \times ( n - m ) + {\sum }_{{m}_{1} = 1}^{M} {\sum }_{{m}_{2} = 1}^{M} {k}_{2} ( {m}_{1} , {m}_{2} ) \times ( n - {m}_{1} ) \times ( n - {m}_{2} ) + L , \qquad ( 1 )$$

where x(n) is the input and y(n) is the output of the system and M is the memory of the system, while the Volterra kernels (kₒ, k₁, k₂, . . . ) describe the dynamics of the system from a hierarchy of system nonlinearities. The kernel values obtained up to a maximum lag M (kernel memory) are combined to form a real symmetric (M+1)×(M+1) square matrix:

$$Q = [ \begin{matrix}{k}_{0} & \frac{1}{2} {k}_{1} ( 1 ) & \frac{1}{2} {k}_{1} ( 2 ) & L & \frac{1}{2} {k}_{1} ( M ) \\ \frac{1}{2} {k}_{1} ( 1 ) & {k}_{2} ( 1 , 1 ) & {k}_{2} ( 1 , 2 ) & L & {k}_{2} ( 1 , M ) \\ \frac{1}{2} {k}_{1} ( 2 ) & {k}_{2} ( 2 , 1 ) & {k}_{2} ( 2 , 2 ) & L & {k}_{2} ( 2 , M ) \\ M & M & M & O & M \\ \frac{1}{2} {k}_{1} ( M ) & {k}_{2} ( M , 1 ) & {k}_{2} ( M , 2 ) & L & {k}_{2} ( M , M )\end{matrix} ] \qquad ( 2 )$$

that can be used to express the second-order Volterra model response, y₂(n) in a quadratic form:

y₂(n)=x^{T}(n)Qx(n), (3)

where T denotes the “transpose” and the (M+1)-dimensional vector x^{T}(n)=[1x(n)x(n−1)Lx(n−M)] is composed of the stimulus M-point epoch at each time n and the constant 1 allows incorporation of the lower order kernel contributions in Eq. (3).

Expansion of the Volterra kernels on a complete basis {βⱼ(m)} transforms Eq. (1) into the multinomial expression

$$y ( n ) = {c}_{0} + {\sum }_{j = 0}^{L - 1} {c}_{1} ( m ) {υ}_{j} ( n ) + {\sum }_{{j}_{1} = 1}^{L - 1} {\sum }_{{j}_{2} = 1}^{L - 1} {c}_{2} ( {j}_{1} , {j}_{2} ) {υ}_{j 1} ( n ) {υ}_{j 2} ( n ) + L \qquad ( 4 )$$

where

$${u}_{j} ( n ) = {\sum }_{m = 0}^{M - 1} {β}_{j} ( m ) \times ( n - m ) \qquad ( 5 )$$

and L is the number of Laguerre functions used. {βⱼ(m)} are the Laguerre functions calculated with Laguerre coefficient α=0.5. Thus, Q can be constructed with the estimated kernels {c₀, c₁, c₂} in the following way:

$$Q = ( \begin{matrix}{c}_{0} & \frac{1}{2} {c}_{1}^{T} {B}^{T} \\ \frac{1}{2} {Bc}_{1} & {B}^{T} {c}_{2} B\end{matrix} ) \qquad ( 6 )$$

where B=[β₀^{T}β₁^{t}Lβ_{L-1}^{T}].

Laguerre functions are preferred as appropriate basis functions because they exhibit exponential decaying properties that make them suitable for physiological system modeling. In addition, due to basis function expansion, the estimation accuracy is maintained even with a small data set length. We have previously shown that a data set with length ˜250 points is sufficient for accurate kernel estimation using the Laguerre expansion⁴²

Because Q is a real symmetric square matrix, there always exists an orthonormal matrix R such that Q=R^{T}ΛR, leading to the expression

y₂(n)=u^{T}(n)Λu(n), (6)

where Λ is the diagonal eigenvalue matrix, and

u(n)=Rx(n) (7)

is the vector of transformed inputs by the orthonormal eigenvector matrix R. Inspection of the real eigenvalues in A allows selection of the significant ones on the basis of relative magnitude (a selection that calls for appropriate threshold criteria) and subsequent selection of the corresponding orthonormal eigenvectors that become the PDMs of this system.

For each significant eigenvalue λᵢ, the values of the corresponding eigenvector μᵢ^{T}=[μ_{i,1}μ_{i,2}Lμ_{i,M+1}] (with the exception of μ_{i,1}) define the ith PDM:

$${p}_{i} ( m ) = {\sum }_{j = 1}^{M + 1} {μ}_{i , j} δ ( m - i + 1 ) , \qquad ( 8 )$$

where δ() denotes the discrete impulse function (Kronecker delta). The obtained ith PDM generates the ith mode output uᵢ(n) via convolution with the stimulus x(n). Note that a constant offset value β₁=μ_{i,0} must be added to the ith mode output uᵢ to express the second-order model prediction ŷ₂ using J PDMs:

$${\hat{y}}_{2} ( n ) = {\sum }_{i = 1}^{J} {{λ}_{i} [ {u}_{i} ( n ) + {β}_{i} ]}^{2} . \qquad ( 9 )$$

Nonzero offset values {βᵢ} give rise to linear terms in terms of {uᵢ} in the model output equation. Note that the matrix Q is not positive definite and, therefore, negative and positive eigenvalues are possible.

In practice, the selection of the significant eigenvalues/eigenvectors must take into account signal-to-noise ratio (SNR) considerations (i.e., setting the selection threshold higher for lower SNR) and trade-offs between prediction accuracy and model complexity. A simple selection criterion is used in this study whereby the selected eigenvalues cumulatively account for at least 90% of the output signal power.

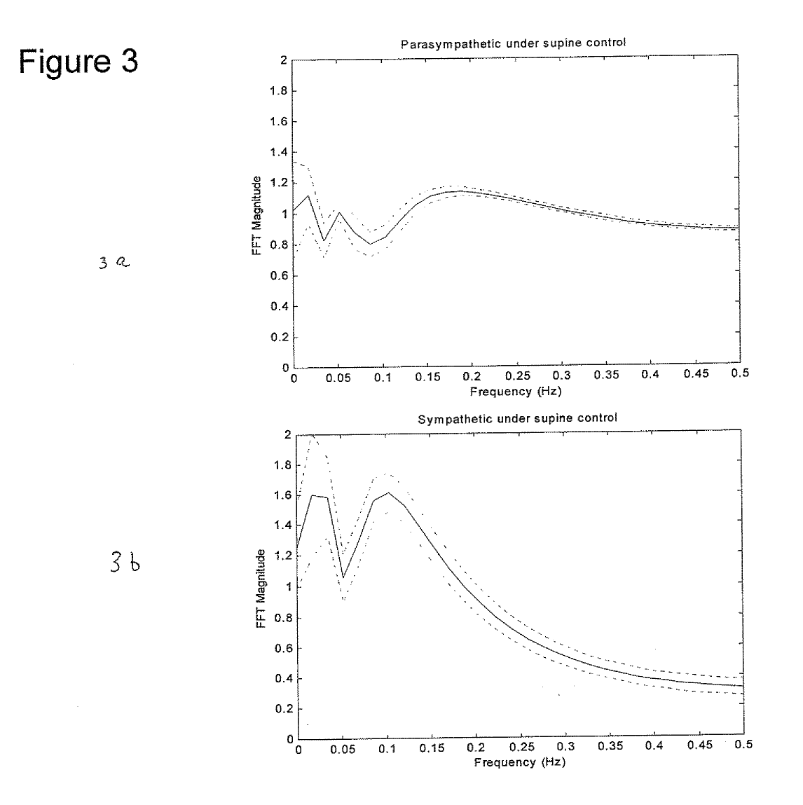

The PDM algorithm provides two PDMs (using M=60, α=0.5, L=6) that correspond to the main frequency-response characteristics of the two autonomic nervous systems, sympathetic and parasympathetic. This procedure yields consistent waveforms corresponding to parasympathetic and sympathetic nervous activities for subjects in the supine position and upright position. In FIG. 3, the solid lines represent average waveforms based on nine subjects with dotted lines corresponding to the standard error bounds. The left and right panels of FIG. 3 show frequency responses of the two PDMs obtained during the control condition; they correspond to the dynamics of the parasympathetic and sympathetic nervous systems, respectively. The dominant peak of the left panel of FIG. 3 is centered at 0.17 Hz, which is in the prescribed frequency range of the parasympathetic nervous system. Furthermore, this PDM also shows a prominent second peak centered at 0.03 Hz. The significance of these two peaks is that many studies have shown that the parasympathetic nervous system operates both in low and high frequency bands. Therefore, this PDM correctly exhibits both the low and high frequencies of the parasympathetic nervous activity. The right panel of FIG. 3 shows prominent peaks in the prescribed frequency band of the sympathetic nervous system. Therefore, we conjecture that the two dominant PDMs correspond to sympathetic and parasympathetic activities. This conjecture, allows separation of the two nervous activities that are known to interact nonlinearly.

To quantitatively obtain the individual activities of the sympathetic and parasympathetic activities obtained by PDMs we integrate the areas of the waveforms corresponding to the sympathetic and parasympathetic activities. In one preferred embodiment the PDMs will be estimated while the subject's cardiovascular system is properly regulated (i.e. through proper medication), that is, during or soon after a subject's visit to his/her physician, that will serve as a means (i.e. threshold) to evaluate instability and/or guide therapy; that is in a preferred embodiment, the threshold value will be unique for every patient.

In a preferred embodiment premature ventricular contractions (PVCs) will be excluded from the analysis as previously described⁴³. In another preferred embodiment premature atrial ventricular contractions (PACs) will be excluded from the analysis using a similar approach with that described for PVCs⁴³.

Thus, a beat will be classified as good when both of the following criteria are satisfied: (i) the morphology criterion, which required the correlation coefficient between the current beat and the average beat to be greater than 0.95, and (ii) the RR criterion, which required that the current RR interval be less than 25% premature or delayed from the mean RR interval of the previous ten beats. If the morphology criterion is not satisfied for a beat (i.e. that is a beat classified as a PVC or PAC) then that and the next beat (and their corresponding RR intervals) will be classified as bad ones. If the RR criterion is not satisfied for a beat (i.e. an aberrantly conducted sinus beat due to bundle branch block) then only that (and its corresponding RR interval) will classified as a bad beat. After all bad beats are discarded, the good beats will be shifted forward so as to form a normal to normal sequence of beats. In another preferred embodiment, given the association of PVCs with increased sympathetic tone¹, PVCs will be included in the analysis.

In another preferred embodiment the method is used to assess the degree of diabetic autonomic neuropathy, the depth of anesthesia, the degree of congestive heart failure, baroreflex sensitivity, renovascular hypertension, chronic orthostatic intolerance, chronic fatigue syndrome, abnormal atrial activity, abnormal brain activity.

Since a subject's increased physical activity is correlated with his/her increased sympathetic tone, in one preferred embodiment the physical activity of the subject will be measured and recorded. In another preferred embodiment the incidence of myocardial ischemia is measured and recorded using either electrocardiographic or (bio)chemical and/or metabolic markers in the blood. In a particularly preferred embodiment these measurements and recordings will be performed by an implantable device (a pacemaker or an ICD or a monitor).

In a preferred embodiment atrial activity reflected in P-to-P (PP) wave variability will be measured and recorded. In another preferred embodiment, if PP wave variability is recorded, PACs will be included in the analysis.

In another preferred embodiment the blood pressure of a cardiac chamber or the vasculature (i.e. arterial pressure) will be measured and recorded and utilized as a means of improving the accuracy of the AI estimation (sympathetic and parasympathetic activity). In another preferred embodiment the instantaneous lung volume will be measured and recorded and utilized as a means of improving the accuracy of the AI estimation (sympathetic and parasympathetic activity).

In another preferred embodiment the PP variability, blood pressure and instantaneous lung volume will be measured by an implanted device. In another preferred embodiment the PP variability, blood pressure and instantaneous lung volume will be measured by means outside the body.

In a preferred embodiment in which in addition to electrocardiographic, beat-to-beat blood pressure and instantaneous lung volume data are also available, the PDM will be calculated from a modification of Eq. (1), which will specifically account for these two additional physiological variables:

$$y ( n ) = {k}_{0} + {\sum }_{m = 1}^{M} {k}_{11} ( m ) hr ( n - m ) + {\sum }_{m = 1}^{M} {k}_{12} ( m ) bp ( n - m ) + {\sum }_{m = 1}^{M} {k}_{13} ( m ) ilv ( n - m ) + {\sum }_{{m}_{1} = 1}^{M} {\sum }_{{m}_{2} = 1}^{M} {k}_{21} ( {m}_{1} , {m}_{2} ) hr ( n - {m}_{1} ) hr ( n - {m}_{2} ) + {\sum }_{{m}_{1} = 1}^{M} {\sum }_{{m}_{2} = 1}^{M} {k}_{22} ( {m}_{1} , {m}_{2} ) bp ( n - {m}_{1} ) bp ( n - {m}_{2} ) + {\sum }_{{m}_{1} = 1}^{M} {\sum }_{{m}_{2} = 1}^{M} {k}_{23} ( {m}_{1} , {m}_{2} ) ilv ( n - {m}_{1} ) ilv ( n - {m}_{2} ) + {\sum }_{{m}_{1} = 1}^{M} {\sum }_{{m}_{2} = 1}^{M} {k}_{24} ( {m}_{1} , {m}_{2} ) hr ( n - {m}_{1} ) bp ( n - {m}_{2} ) + {\sum }_{{m}_{1} = 1}^{M} {\sum }_{{m}_{2} = 1}^{M} {k}_{25} ( {m}_{1} , {m}_{2} ) hr ( n - {m}_{1} ) ilv ( n - {m}_{2} ) + {\sum }_{{m}_{1} = 1}^{M} {\sum }_{{m}_{2} = 1}^{M} {k}_{26} ( {m}_{1} , {m}_{2} ) bp ( n - {m}_{1} ) ilv ( n - {m}_{2} ) + … \qquad ( 10 )$$

where hr, bp and ilv in Eq. (10) denote variability of the instantaneous heart rate (hr), blood pressure and instantaneous lung volume variability, respectively. The second and last rows of Eq. (10) represent 2^{nd}-order self and cross terms between these three physiological variables, respectively. The Q matrix in Eq. (2) will then be changed to include all of the “k” terms in Eq. (10). The rest of steps for the calculation of the PDM is the same as those outlined earlier.

In one preferred embodiment the values of statistical indices describing physiological signal recordings like the RR, physical activity, presence of ischemia, blood pressure and instantaneous lung volume will be stored, updated periodically and be also used as thresholds besides the patient's baseline values, to trigger therapy. Specifically, such values of these indices will be correlated with a time window prior to the initiation and/or after termination of abnormal cardiac activity. In a preferred embodiment indices for physical activity and presence of ischemia will be used to normalize the estimation of sympathetic and parasympathetic activity indices and to trigger therapy.

In one preferred embodiment when the patient specific criteria are indicative of a ventricular tachy-arrhythmia event to occur soon, these measurements of beat-to-beat cardiac activity will be used to guide therapy. In another preferred embodiment the patient specific criteria to guide therapy may be indicative of a ventricular brady-arrhythmia.

In a preferred embodiment chemical substance therapy will be initiated first, in a controlled manner and at specified times. In another preferred embodiment the patient's one or more electrodes placed in or on the patient's heart suitable for delivering electrical impulses to the heart (“pacing electrodes” or “active electrodes”) will be used to guide electrical therapy to alter the beat-to-beat electrical activity of the heart. The sensing electrodes are connected by leads to a device which processes the electrical signals and which is also connected by leads to the electrodes used for delivering electrical impulses to the heart. Some electrodes may serve as both “sensing electrodes” and “pacing electrodes”.

In a preferred embodiment the ICD is coupled to (i) a right ventricular lead for sensing and/or delivering pacing or shocking pulses to the right ventricle, (ii) a right atrial lead for sensing and/or delivering pacing or shocking pulses to the right atrium and (iii) a lead in the coronary sinus for sensing and/or delivering pacing or shocking pulses alone or in conjunction with the ventricular or atrial lead, (iv) a left ventricular lead for sensing and/or delivering pacing or shocking pulses to the left ventricle,

In a preferred embodiment, all physiological parameters pertinent to this application will be recorded and the corresponding indices of interest quantified, while the subject's underlying condition is well balanced and regulated following a specialist's orders. These parameters and indices will serve as a custom baseline, against which subsequent measurements and estimates will be compared. In a preferred embodiment, each estimate will have a different threshold above or below which appropriate therapy will be triggered. In another preferred embodiment, that threshold will be adjusted for each subject in order to provide optimal balance of quality of life (that is dependent, for example, on the subject's condition, medications, etc.) and the need for appropriate therapy, versus the frequency of provided therapy.

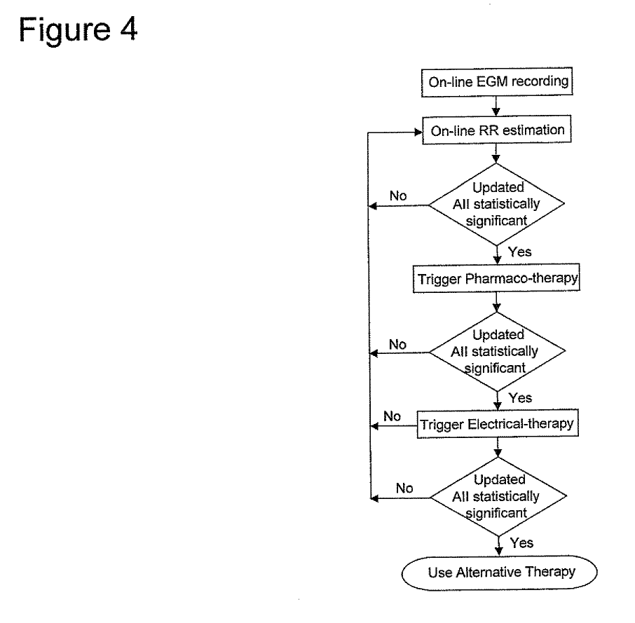

In a preferred embodiment the methodology in restoring autonomic system imbalances is presented in FIG. 4. In one preferred embodiment the AI will be estimated by estimating the ratio of the sympathetic to parasympathetic indices derived from the PDM. In one preferred embodiment if significant changes (defined above) in the AI is present, for example if that index is found to exceed a patient-specific threshold value, for an interval of at least 2 min, than the device starts delivering a drug in the heart to alter that AI. Then, following the delivery of the drug the device will determine the degree to which the statistical properties of the PDM estimated spectra as well as the sympathetic/parasympathetic ratio derived from the PDM from which the AI is derived subsequent to the drug delivery, match those preceeding the drug delivery over some period of time (for example, 2 min). The device then will adaptively adjust the drug flow in such a way to reduce variation in the AI from that measured during baseline. In one preferred embodiment, the device adjusts the drug flow based on a determination of the mean beat-to-beat variation in the AI over multiple (for example, 3) periods of time (for example, 2 min each). In this manner the device will not attempt to track variation in the AI that may, for example, be due to extraneous sources such as respiration rather than being due to variation in intrinsic cardiac conduction processes, because the period of time is sufficiently long to average over several respiratory cycles.

In one preferred embodiment, if after the drug infusion significant AI is present, then the device will start pacing the heart at a mean (μ) RR rate that is an increment above the resting RR rate, but not at an RR rate to exceed an upper heart rate limit specified for the individual patient. The increment may be approximately ˜2-15% of resting RR rate or more if needed to achieve one-to-one capture of the patients' ventricles. The device will pace the heart using a non-equally spaced “adaptive pattern” of pulses, that is, will adaptively lengthen or shorten by a small increment, for example, 5 milliseconds, and for some period of time, for example 2 minutes, in order to restore the value of that AI to its physiological value.

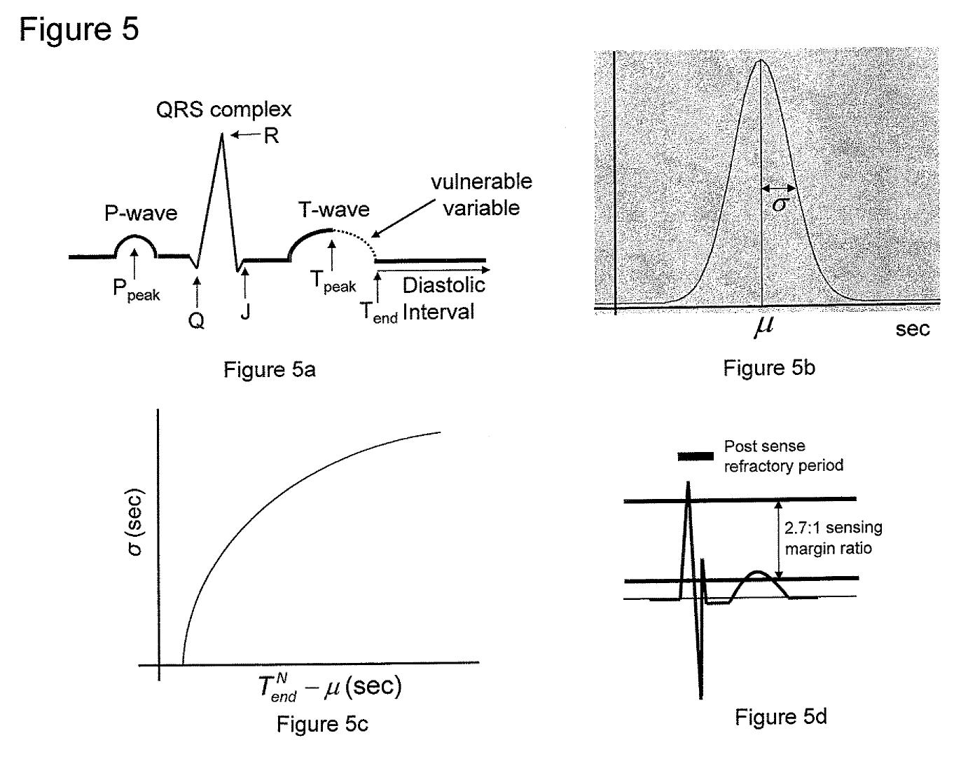

In a preferred embodiment the μ RR rate will be determined using a standard R-wave peak detection algorithm and pacing pulses will be initially applied in such a way that μ is not smaller than the RT_{end} (FIG. 5a) in order to avoid delivering pacing pulses in the vulnerable period of the T-wave. Determination of μ will be obtained from the statistical properties of the PDM estimated spectra as well as the sympathetic/parasympathetic ratio derived from the PDM in order to match those preceding the delivery of the pacing pulses over some period of time (for example, 2 min).

In a preferred embodiment, in order to ensure the rapid application of electrical therapy during the diastolic interval (FIG. 5a), the sensing algorithm of the programmable-stimulator will make use of an autogain algorithm that has been demonstrated to have a clear advantage over fixed gain sensing algorithms⁴⁴. Following detection of the QRS complex a “post sense refractory period” will be employed. Then, the sensing algorithm using a different sensitivity (i.e. 2.7:1) with respect to the QRS complex will identify the end of the T-wave and trigger electrical therapy (as described above) upon return of the T-wave to baseline.

For example, if lengthening the inter-impulse interval increases the discrepancy between the currently estimated AI and the baseline one, then the device will shorten the subsequent inter-impulse interval. Conversely, if lengthening the inter-impulse interval decreases the discrepancy in the AI with respect to the baseline, then the device will further shorten the next inter-impulse interval. In this manner the device will adaptively adjust the inter-beat interval in such a way to reduce variation in the AI with respect to the baseline. Thus, following the delivery of each pacing pulse the device will determine the degree to which the AI subsequent to a given electrical impulse matches the AI value preceding that given electrical impulse.

Pacing stimuli will be delivered during the non-vulnerable period during the cardiac cycle in order to avoid inducing ventricular fibrillation, and preferably during the diastolic interval defined as the present RR interval minus the AP duration of the previous beat. In one preferred embodiment this may be accomplished by cross-correlating the T wave subsequent to an electrical impulse with the preceding T wave. The time base for each T wave is measured relative to the time of the electrical impulse which precedes it. The cross correlation procedure allows one to determine the cross correlation coefficient as well as the offset in time of the T wave subsequent to a given electrical impulse compared to the T wave preceding it.

In one preferred embodiment the methodology of the PDM is applied in electrocardiographic indices such as the (i) PₚₑₐₖR, (ii) PₚₑₐₖPₚₑₐₖ, (iii) QT_{end}, (iv) RT_{end}, (v) JT_{end}, (vi) QTₚₑₐₖ (vii) RTₚₑₐₖ intervals.

### REFERENCES

1. Priori S G, Corr P B. Mechanisms underlying early and delayed afterdepolarizations induced by catecholamines. Am J Physiol. June 1990; 258(6 Pt 2):H1796-1805.
2. Levy M N. Sympathetic-parasympathetic interactions in the heart. In: Kulbertus H E, Frank F, eds. Neurocardiology. Mt. Kisco, N.Y.: Futura Publishing Co; 1988:85-98.
3. Levy M N, martin P J. Autonomic neural control of cardiac function. In: Sperelakis N, ed. Physiology and Pathophysiology of the Heart. 3rd ed. Boston, Mass.: Kluwer Academic Publishers; 1995:413-430.
4. Yang T, Senturia J B, Levy M N. Antecedent sympathetic stimulation alters time course of chronotropic response to vagal stimulation in dogs. Am J Physiol. April 1994; 266(4 Pt 2):H1339-1347.
5. Yang T, Levy M N. Effects of intense antecedent sympathetic stimulation on sympathetic neurotransmission in the heart. Circ Res. January 1993; 72(1):137-144.
6. Yang T, Levy M N. Sequence of excitation as a factor in sympathetic-parasympathetic interactions in the heart. Circ Res. October 1992; 71(4):898-905.
7. Eckberg D L, Drabinsky M, Braunwald E. Defective cardiac parasympathetic control in patients with heart disease. N Engl J Med. Oct. 14, 1971; 285(16):877-883.
8. Cohn J N, Levine T B, Olivari M T, et al. Plasma norepinephrine as a guide to prognosis in patients with chronic congestive heart failure. N Engl J Med. Sep. 27, 1984; 311(13):819-823.
9. Tovar O H, Jones J L. Electrophysiological Deterioration During Long-Duration Ventricular Fibrillation. Circulation. 2000; 102:2886-2891.
10. Bristow M R, Ginsburg R, Minobe W, et al. Decreased catecholamine sensitivity and beta-adrenergic-receptor density in failing human hearts. N Engl J Med. Jul. 22, 1982; 307(4):205-211.
11. Hjalmarson A, Goldstein S, Fageberg B, et al. Effect of metoprolol CR/XL in chronic heart failure: Metoprolol CR/XL Randomised Intervention Trial in Congestive Heart Failure (MERIT-HF). Lancet. Jun. 12, 1999; 353(9169):2001-2007.
12. Kuck K H, Cappato R, Siebels J, et al. Randomized comparison of antiarrhythmic drug therapy with implantable defibrillators in patients resuscitated from cardiac arrest: the Cardiac Arrest Study Hamburg (CASH). Circulation. Aug. 15, 2000; 102(7):748-754.
13. Krumholz H M, Radford M J, Wang Y, et al. National use and effectiveness of beta-blockers for the treatment of elderly patients after acute myocardial infarction: National Cooperative Cardiovascular Project. Jama. Aug. 19, 1998; 280(7):623-629.
14. Pagani M, Montano N, Porta A, et al. Relationship between spectral components of cardiovascular variabilities and direct measures of muscle sympathetic nerve activity in humans. Circulation. Mar. 18, 1997; 95(6):1441-1448.
15. van de Borne P, Montano N, Zimmerman B, et al. Relationship between repeated measures of hemodynamics, muscle sympathetic nerve activity, and their spectral oscillations. Circulation. Dec. 16, 1997; 96(12):4326-4332.
16. Wolf M M, Varigos G A, Hunt D, et al. Sinus arrhythmia in acute myocardial infarction. Med J Aust. 1978; 2(2):52-53.
17. Bigger J T, Fleiss J L, Rolnitzky L M, et al. Stability over time of heart period variability in patients with previous myocardial infarction and ventricular arrhythmias. The CAPS and ESVEM investigators. Am J. Cardiol. 1992; 69(8):718-723.
18. Fallen E L, Kamath M V, Ghista D N. Power spectrum of heart rate variability: a non-invasive test of integrated neurocardiac function. Clin Invest Med. 1988; 11(5):331-340.
19. Kleiger R E, Miller J P, Bigger J T, et al. Decreased heart rate variability and its association with increased mortality after acute myocardial infarction. Am J Cardiol. 1987; 59(4):256-262.
20. Malik M, Farrell T, Cripps T, et al. Heart rate variability in relation to prognosis after myocardial infarction: selection of optimal processing techniques. Eur Heart J. 1989; 10(12):1060-1074.
21. Adamson P B, Huang M H, Vanoli E, et al. Unexpected interaction between beta-adrenergic blockade and heart rate variability before and after myocardial infarction. A longitudinal study in dogs at high and low risk for sudden death. Circulation. 1994; 90(2):976-982.
22. Electrophysiology TfotEsocatNASoPa. Heart rate variability, standards of measurement, physiological interpretation and clinical use. Circulation. 1996; 93:1043-1065.
23. Bigger J T, Rolnitzky L M, Steinman R C, et al. Predicting mortality after myocardial infarction from the response of RR variability to antiarrhythmic drug therapy. J Am Coll Cardiol. 1994; 23(3):733-740.
24. Huikuri H V, Makikallio T H, Airaksinen K E, et al. Power-law relationship of heart rate variability as a predictor of mortality in the elderly. Circulation. 1998; 97(20):2031-2036.
25. Malik M, Xia R, Odemuyiwa O, et al. Influence of the recognition artefact in automatic analysis of long-term electrocardiograms on time-domain measurement of heart rate variability. Med Biol Eng Comput. 1993; 31(5):539-544.
26. Schmidt G, Monfill G E. Nonlinear methods for heart rate variability. In: Malik M, Camm A J, eds. Heart Rate Variability. Armonk, N.Y.: Futura; 1995:87-98.
27. Kanters J K, Hojgaard M V, Agner E, et al. Short- and long-term variations in non-linear dynamics of heart rate variability. Cardiovasc Res. 1996; 31(3):400-409.
28. Kanters J K, Holstein-Rathlou N H, Agner E. Lack of evidence for low-dimensional chaos in heart rate variability. J Cardiovasc Electrophysiol. 1994; 5(7):591-601.
29. Grassberger P, Procaccia I. Measuring the strangeness of strange attractors. Physica D. 1983; 9:183-208.
30. Tapanainen J M, Thomsen P E, Kober L, et al. Fractal analysis of heart rate variability and mortality after an acute myocardial infarction. Am J. Cardiol. Aug. 15, 2002; 90(4):347-352.
31. Yang C C, Kuo T B. Assessment of cardiac sympathetic regulation by respiratory-related arterial pressure variability in the rat. J Physiol. Mar. 15, 1999; 515 (Pt 3):887-896.
32. Bianchi M, Mainardi L T, Carloni S, et al. Continuous monitoring of the sympatho-vagal balance through spectral analysis. IEEE Eng. Med. Biol. September/October 1997; 16:64-73.
33. Zaza A, Lombardi F. Autonomic indexes based on the analysis of heart rate variability: a view from the sinus node. Cardiovasc Res. June 2001; 50(3):434-442.
34. Stevenson W G, Stevenson L W. Prevention of Sudden Death in Heart Failure. J Cardiovasc Electrophysiol. 2001; 12(1):112-114.
35. Prystowsky E N. Screening and therapy for patients with nonsustained ventricular tachycardia. Am J. Cardiol. 2000; 86(9 Suppl 1):K34-K39.
36. Buxton A E, Duc J, Berger E E, et al. Nonsustained ventricular tachycardia. Cardiol Clin. 2000; 18(2):327-336, viii.
37. Buxton A E, Lee K L, DiCarlo L, et al. Electrophysiologic testing to identify patients with coronary artery disease who are at risk for sudden death. Multicenter Unsustained Tachycardia Trial Investigators. N Engl J Med. 2000; 342(26): 1937-1945.
38. Bigger J T, Fleiss J L, Steinman R C, et al. Frequency domain measures of heart period variability and mortality after myocardial infarction. Circulation. 1992; 85(1):164-171.
39. Fallen E L, Kamath M V, Ghista D N, et al. Spectral analysis of heart rate variability following human heart transplantation: evidence for functional reinnervation. J Auton Nerv Syst. 1988; 23(3):199-206.
40. Chon K H, Yip K H, Camino D J, et al. Modeling nonlinear determinism in short time series from noise driven discrete and continuous systems. Int J Bifurcation & Chaos. 2000; 10:2745-2766.
41. Tarvainen M P, Ranta-Aho P O, Karjalainen P A. An advanced detrending method with application to HRV analysis. IEEE Trans Biomed Eng. February 2002; 49(2): 172-175.
42. Marmarelis V Z, Chon K H, Chen Y M, et al. Nonlinear analysis of renal autoregulation under broadband forcing conditions. Ann Biomed Eng. 1993; 21(6):591-603.
43. Armoundas A A, Ju K, Iyengar N, et al. A stochastic nonlinear autoregressive algorithm reflects nonlinear dynamics of heart-rate fluctuations. Ann Biomed Eng. February 2002; 30(2):192-201.
44. Sperry R E, Ellenbogen K A, Wood M A, et al. Failure of a second and third generation implantable cardioverter defibrillator to sense ventricular tachycardia: implications for fixed-gain sensing devices. Pacing Clin Electrophysiol. May 1992; 15(5):749-755.

What is claimed is:

1. A method for quantifying autonomic system disturbances comprising:
detecting one or more physiological parameters;
measuring beat-to-beat variability in the one or more physiological parameters; and
quantifying the instantaneous sympathetic and parasympathetic components of the autonomic system.

2. The method of claim 1, wherein the one or more physiological parameters is selected from the group consisting of cardiac ventricular electrical activity, blood pressure activity, instantaneous-lung-volume activity, and any combination thereof.

3. (canceled)

4. (canceled)

5. The method of claim 1, wherein results from the step of quantifying are used to control therapy to reduce the likelihood of heart rhythm disturbances occurring.

6. The method of claim 2, wherein the beat-to-beat variability is determined from intervals selected from the group consisting of RR intervals QT_{end} intervals, RT_{end} intervals, JT_{end} intervals, QTₚₑₐₖ intervals, RTₚₑₐₖ intervals and any combination thereof.

7-11. (canceled)

12. The method of claim 5, wherein the therapy comprises one or both of the delivery of one or more chemical substances and the delivery of electrical impulses to the heart.

13. (canceled)

14. The method of claim 12, wherein the electrical impulses are controlled to alter one or both of the RR interval and the diastolic interval.

15. (canceled)

16. The method of claim 1, wherein a system disturbance is tachyarrhythmia or bradyarrhythmia.

17-18. (canceled)

19. The method of claim 1, wherein the measuring step is performed in an implanted device.

20. The method of claim 5, wherein the therapy is delivered by an implanted device.

21-27. (canceled)

28. Apparatus for the prevention of a heart rhythm disturbance, comprising:
a cardiac electrical activity detector;
circuitry that receives and measures beat-to-beat variability within the cardiac electrical activity;
means that estimates the AI from beat-to-beat variability within the cardiac electrical activity; and
a control means that accepts the measured beat-to beat variability and controls the delivery of therapy to reduce the likelihood of a heart rhythm disturbance.

29. The apparatus of claim 28 for the prevention of a heart rhythm disturbance, comprising:
a blood pressure activity detector;
circuitry that receives and measures beat-to-beat variability within the blood pressure activity;
means that estimates the AI from beat-to-beat variability within the cardiac electrical and blood pressure activities; and
a control means that accepts the measured beat-to beat variability and controls the delivery of therapy to reduce the likelihood of a heart rhythm disturbance.

30. The apparatus of claim 28 for the prevention of a heart rhythm disturbance, comprising:
an instantaneous-lung-volume (respiration) detector;
circuitry that receives and measures beat-to-beat variability within the instantaneous-lung-volume activity;
means that estimates the AI from beat-to-beat variability within the cardiac electrical, blood pressure and instantaneous-lung-volume activities; and
a control means that accepts the measured beat-to beat variability and controls the delivery of therapy to reduce the likelihood of a heart rhythm disturbance.

31. The apparatus of claim 28, wherein the beat-to-beat variability comprises heart rate variability.

32. The apparatus of claim 28, wherein the therapy comprises one or both of the delivery of one or more chemical substances and the delivery of electrical impulses to the heart.

33-35. (canceled)

36. The apparatus of claim 28, wherein the heart rhythm disturbance is a ventricular tachyarrhythmia or a ventricular bradyarrhythmia.

37. (canceled)

38. The apparatus of claim 28, wherein the cardiac electrical activity detector further comprises:
at least one passive electrode within the heart; and electrical activity recording means.

39. The apparatus of claim 28, wherein the circuitry is incorporated with an implanted device.

40. The apparatus of claim 28, wherein the controlled therapy is delivered by an implanted device.

41. (canceled)

42. The apparatus of claim 28, wherein the apparatus comprises an implantable device having means for generating electrical stimulating pulses of specified energies and applying the pulses to body tissue at specified times.

43-45. (canceled)

46. The apparatus of claim 28, further comprising:
an adjuster for adjusting for individual subjects threshold values employed by the circuitry and control means.

47-64. (canceled)
